(19) United States
(12) Patent Application Publication
(10) Pub. No.: US 2014/0107497 A1
(43) Pub. Date: Apr. 17, 2014

(54) NON-INVASIVE VALSALVA MANEUVER (VM) HEART FAILURE DIAGNOSTIC METHOD AND APPARATUS

(71) Applicant: Semler Scientific, Inc, Portland, OR (US)

(72) Inventors: Herbert J. Semler, Portland, OR (US); Shawn Gregory Scott, Portland, OR (US); Robert McRae, San Jose, CA (US)

(73) Assignee: Semler Scientific, Inc, Portland, OR (US)

(21) Appl. No.: 13/652,223

(22) Filed: Oct. 15, 2012

(51) Int. Cl. A61B 5/026; A61B 5/024

(52) U.S. Cl. CPC A61B 5/0261; A61B 5/024
USPC 600/479; 600/508

(57) ABSTRACT

Method and apparatus for diagnosing heart failure are disclosed. They include monitoring a subject's pulsatile blood flow with a non-invasive probe during a Valsalva maneuver (VM), processing data therefrom to calculate fall in flow, hear rate changes, Rebound, and heart stroke volume during the VM. Monitored and calculated results are compared to defined thresholds and interpreted and reported. The apparatus takes the form of a pulsatile blood flow probe on a finger or toe or in a mouthpiece facilitating the VM, the mouthpiece optionally including a pressure transducer or digital monometer to ensure that the subject is performing the VM within required pressure and time ranges. The method and apparatus include a controller or digital processor for processing and reporting the results of the monitoring, calculations, comparisons, interpretation, and reporting of the diagnostic results.

### RELATED APPLICATIONS

This application claims the benefit of priority to U.S. Non-provisional application Ser. No. 12/317,538, filed on 24 Dec. 2008 and entitled BODY COMPOSITION, CIRCULATION, AND VITAL SIGNS MONITOR AND METHOD, and to U.S. non-provisional application Ser. No. 12/001,505 filed on 11 Dec. 2007 and entitled CIRCULATION MONITORING SYSTEM AND METHOD, now U.S. Pat. No. 7,628,760 B2, which claims the benefit of priority to U.S. non-provisional application Ser. No. 11/017,455 filed on 20 Dec. 2004 and entitled NON-INVASIVE BODY COMPOSITION MONITOR, SYSTEM AND METHOD, the contents and disclosures of which are hereby incorporated herein in their entirety by this reference.

### FIELD OF THE INVENTION

The invention relates generally to the field of heart failure diagnostics. More particularly, the invention relates to monitoring a subject's pulsatile blood flow during a so-called valsalva maneuver (VM) to determine susceptibility of the subject to heart failure.

### BACKGROUND OF THE INVENTION

Conventionally, cardiac patient testing has relied on invasive procedures involving cardiac catheterization.

Recently, the assignee of the present invention described and illustrated a non-invasive circulation monitor that analyzes a subject's cardiac pulsatility or flow and calculates a figure of merit called a circulation index (CI) representative of the subject's circulation level to diagnose peripheral artery disease (PAD). That invention subject to common assignment and ownership with the present application is described in U.S. Pat. No. 7,628,760 B2 entitled CIRCULATION MONITORING SYSTEM AND METHOD and issued Dec. 8, 2009. Familiarity with the monitoring and CI analysis, interpretation, and reporting teachings of that patent is assumed.

### BRIEF DESCRIPTION OF THE DRAWINGS

FIG. 1 is a system block diagram illustrating the use of a blood pulsatility monitor, a Valsalva maneuver (VM) device, and a controller for monitoring a subject's circulation and for deriving therefrom a baseline circulation index (CI), a VM-based fall-in-flow (FiF), heart stroke volume (SV), Rebound, and other useful metrics indicative of the subject's heart failure susceptibility.

FIG. 2 is a flowchart illustrating the calculation of the FiF during the VM, comparison thereof to a defined threshold, and interpretation and reporting of a heart failure diagnosis based thereon.

FIG. 3 is a flowchart illustrating the calculation of a heart rate ratio (HRR) between a baseline HR and a VM HR, for comparison to a defined HRR threshold, and interpretation and reporting of a heart failure diagnosis based thereon.

FIG. 4 is a flowchart illustrating the calculation of heart stroke volume (SV) based upon the subject's HRR and FiF, comparison of the SV result with a defined SV threshold, and interpretation and reporting of a heart failure diagnosis based thereon.

FIGS. 5A and 5B illustrate superimposed pulse and CI waveforms respectively contrasting a heart failure subject and a ‘healthy’ subject.

FIG. 6 and associated Details A and B represent isometric and fragmentary side elevations of an apparatus in accordance with another embodiment of the invention that integrates the VM device and optionally a manometer/pressure indicator of FIG. 1 into a disposable mouthpiece and reusable VM device body apparatus to compactly embody a part of the system of FIG. 1.

FIG. 7 illustrates a display part of the system or apparatus of FIG. 1, the display featuring the manometer and pulsatility monitoring results or other pertinent graphic or tabular diagnostic data.

FIGS. 8A and 8B illustrate two versions of a report generated by the invented system, with FIG. 8A featuring a heart failure subject and with FIG. 8B featuring a ‘healthy’ subject.

FIG. 9 is a flowchart illustrating the calculation of the Rebound immediately following the VM, comparison thereof to defined thresholds, and interpretation and reporting of a heart failure diagnosis based thereon.

### DETAILED DESCRIPTION OF THE PREFERRED EMBODIMENTS

Previous heart failure diagnostic procedures have employed the previous ‘gold standard’: catheterization. But catheterization requires a cardiac catheter lab and is complicated, invasive, and expensive.

It has been discovered that heart failure-prone candidate subjects can be reliably and repeatably diagnosed by externally, non-invasively monitoring the subject's blood flow changes during administration to the subject of a Valsalva maneuver (VM). Heart failure-prone subjects show characteristic signatures marked by minimal heart rate change; stable, elevated blood pressure; and a lack of over-shoot (momentarily and dramatically elevated) post-VM recovery response (also termed ‘Rebound’). Thus, the present invention provides a non-invasive technique for accurately diagnosing heart failure.

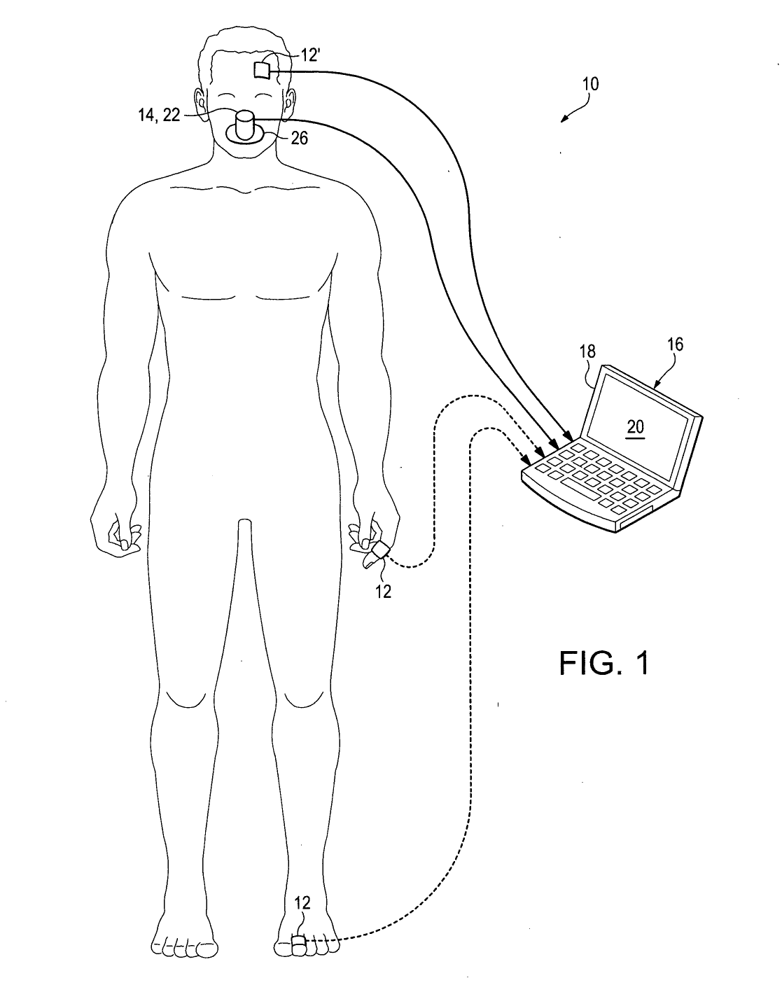

Referring first to FIG. 1, invented system 10 includes a blood flow or pulsatility probe 12 or 12′ operatively affixed to a subject's extremity, e.g. a finger (dashed line), toe (dashed line), earlobe (not shown), or other surface area of the body, e.g. a forehead (solid line), lips (not shown), etc.; a VM device 14 operable to accept a subjects expiratory pressure during pulsatility monitoring; and a controller 16 analyzing and interpreting a subject's cardiac response characteristics and, optionally, parameters from the VM device 14. In accordance with one embodiment of the invention, such analysis includes time correlating and calculating a circulation index (CI) and a change in CI or fall in flow (FiF) during the VM interval, for comparing the FiF to a defined threshold value, and for reporting a diagnostic result based thereon. Those of skill will appreciate that forehead probe 12′ is a different physical embodiment of finger/toe probes 12 or 12′ described and illustrated in detail in referenced U.S. Pat. No. 7,628,760 B2, with a tiny generally planar printed circuit board (PCB) or flex-circuit mounting the optical and electronic devices along with a wireless communications means or a connector/cable combination if wired as shown.

Those of skill in the art will appreciate that the pulsatility monitoring and derivation therefrom of the subject's CI may be made in any suitable manner such as that described in detail in the above-referenced U.S. Pat. No. 7,628,760 B2, which patent enjoys common ownership with the present application. Alternative methods of determining a circulation figure of merit are contemplated as being within the spirit and scope of the invention.

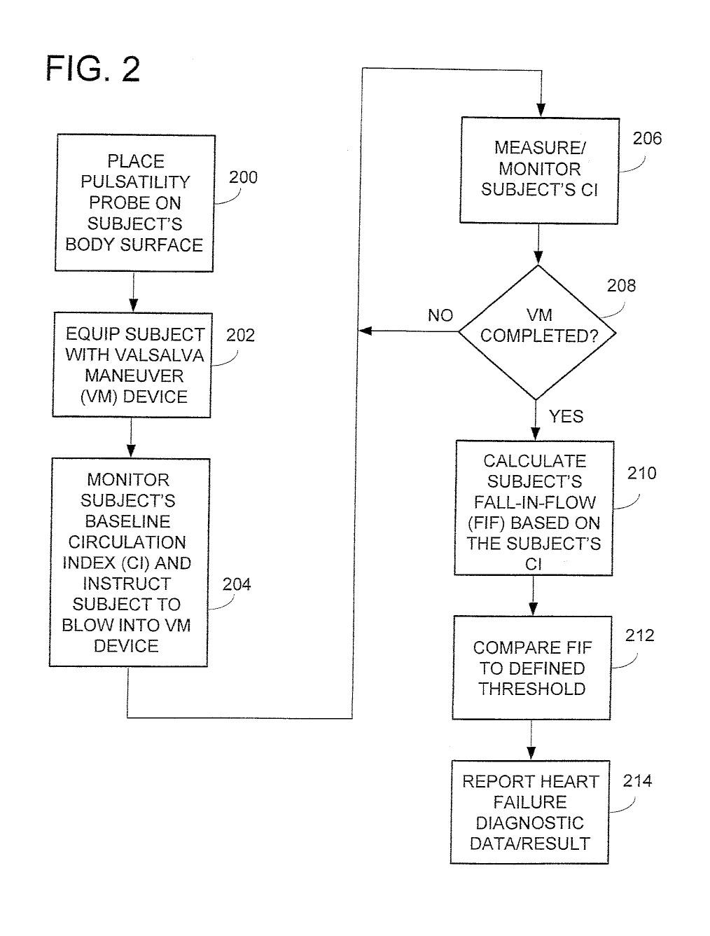

FIG. 2 illustrates the invented method in one embodiment by which controller 16 analyzes the inputs from pulsatility probe 12 and VM device 14 to diagnose a subject's susceptibility to heart failure. The method includes at 200 placing a pulsatility probe on an extremity or other area of the surface of a subject's body. The probe is operatively coupled with a controller configured to calculate a continuous or discrete circulation index (CI) throughout a baseline interval before, during, and after a VM. At 202, the subject is equipped with a VM device including a mouthpiece into which to blow in accordance with an instructed protocol that includes a sustained period of time, e.g. approximately fifteen seconds, and a pressure range, e.g. approximately forty millimeters of mercury (40 mm Hg). At 204, the subject's baseline CI is monitored for a defined period of time, and the subject is instructed continuously and steadily to blow into the VM device for a specific period of time, e.g. fifteen seconds.

At 206, the controller measures the subject's CI during the VM interval. At 208, it is determined whether the VM has been completed. If not, then the measuring/monitoring of the subject's CI continues at 206. When the VM is completed, then at 210, the controller calculates the subject's so-called fall-in-flow (FiF) based on the maximum difference between the baseline CI and minimum CI during the VM interval. Thereafter, at 212, the FiF during the VM interval corresponding to the subject's cardiac response is compared to a defined threshold level, e.g. between approximately 0.05 and approximately 0.20, preferably approximately 0.13 (corresponding to between approximately 5% and approximately 20%, preferably approximately 13% in percentages). If the subject's FiF is less than the defined threshold, then the result of the subject's test is interpreted as being positive for heart failure. Finally, at 214, the result of the heart failure test is displayed, printed, or otherwise reported.

The subject's CI or equivalent indicator of cardiac flow capacity in an extremity or area of the surface or otherwise non-invasively accessible region of the body may be calculated in any suitable manner, including the manner described in the referenced U.S. Pat. No. 7,628,760 B2. Broadly speaking, the subject's fall in flow (FiF) over the pertinent VM interval may be calculated in any suitable manner, including the manner described immediately below by resort to the invented method in accordance with a first embodiment of the invention. The invented method of calculating the subject's FiF utilizes the following Equation 1:

FiF=AVG(CI_Baseline)−Min(CI_Valsalva) (1)

wherein AVG(CI_Baseline) is the average CI measured during a defined period of time before the Valsalva maneuver begins, and wherein Min(CI_Valsalva) is the minimum CI during the Valsalva maneuver.

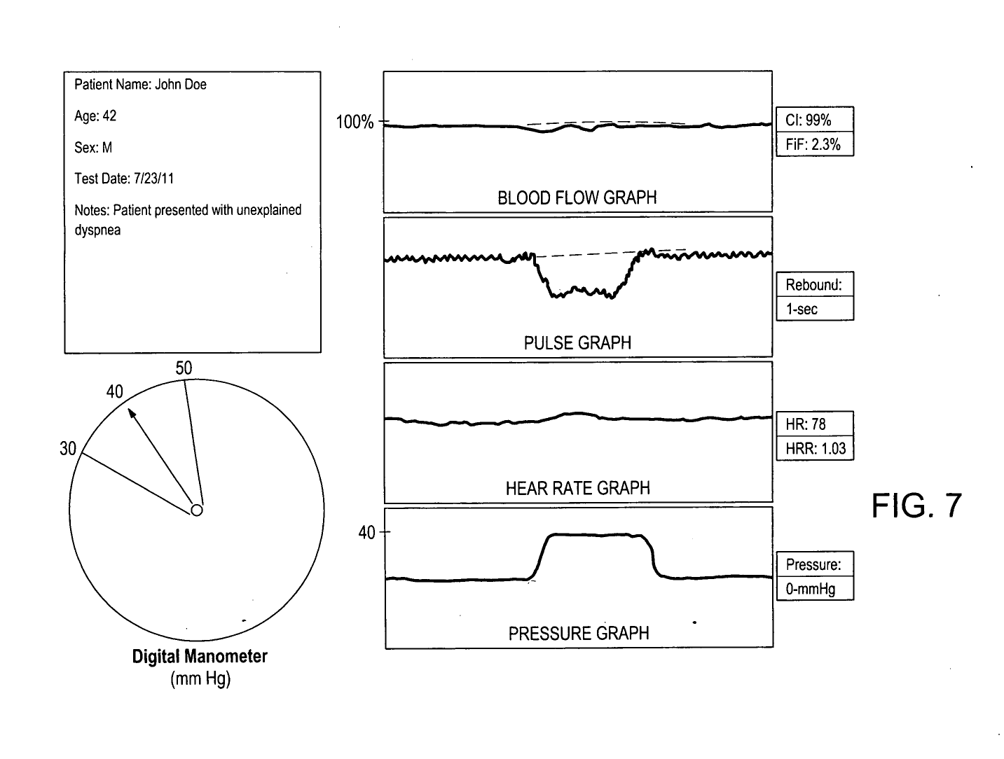

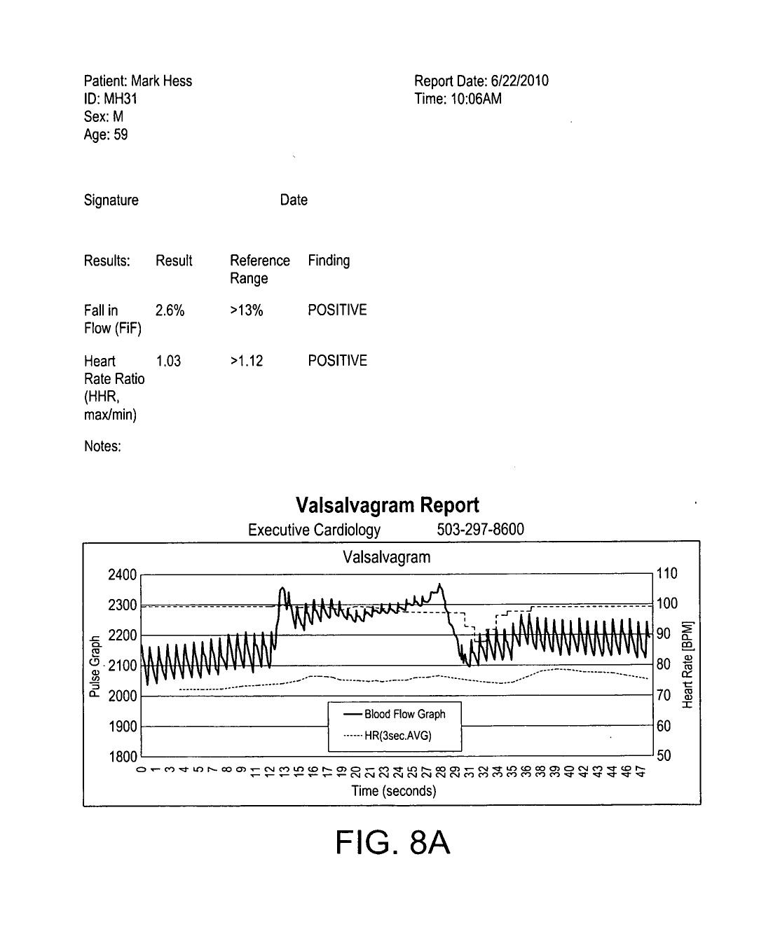

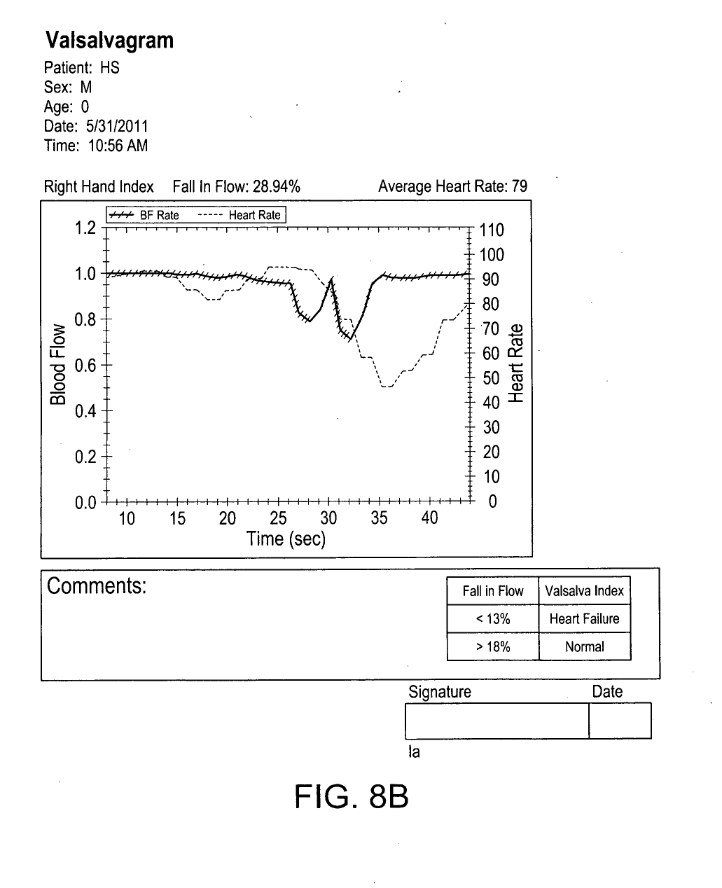

Those of skill in the art will appreciate that the heart failure test may be displayed, printed, or otherwise reported in any suitable manner to any suitable individual or group of individuals. For example, controller 16 may be housed in a portable housing 18 along with a display 20, e.g. an LCD or other flat-screen display or the like, configured to display a manometer reading and/or the CI, and/or the graphic or numeric or color-coded results of the monitoring, calculating, and comparing method steps. These display and print reporting options will be further described below by reference to FIGS. 7, 8A, and 8B.

Optional display 20 also or alternatively may display instructions to the subject or clinician or other attendant on operation of the heart failure diagnostic system. Such instructions or feedback may be in visual display or auditory forms. Instructions may provide feedback to the subject to maintain or change expiratory pressure. Those of skill in the art will appreciate that the software or firmware that implements the monitoring, calculating, and comparing method steps typically resides in a memory that is a part of controller 16 and the software instructions are typically executed within a digital processor, e.g. a microprocessor, within the controller. Alternatives or augmentations to display 20 and a graphical user interface (GUI) presented herein are contemplated for interaction between the system and the subject as being within the spirit and scope of the invention. Such may include an artificially intelligent (AI) audio tutorial that uses voice generation and voice recognition software to enable the controller to ‘talk’ to and ‘listen’ to the subject and to audibly ‘report’ the results of the heart failure diagnostic.

Those of skill in the art will appreciate that controller 16 and display 20 may take any suitable form. For example, they may be parts of any suitable general-purpose or special-purpose (dedicated) computing platform such as a personal computer (PC), laptop computer, notebook computer, tablet, etc. Those of skill in the art will also appreciate that suitably programmed controller 16 in the form of a PC, for example, provides substantial data processing capability to at least semi-automate the process of data collection, processing, analysis, interpretation, and presentation or reporting that form a part of the invented heart failure diagnostic technique.

Those of skill in the art also will appreciate that the controller and display may be embedded in a non-dedicated, portable, hand-held device such as a so-called ‘smart phone’. Installed or hosted or downloaded application software would be licensed or otherwise subscribed to such a smart phone in the form of a so-called ‘app’ that enables the functionality of the controller portion of system 10. For example, the pulsatility or flow probe, VM device, and other operatively connected devices within the spirit and scope of the invention could be rendered in input/output (I/O) form, fit and function as universal serial bus (USB) or BlueTooth or WiFi devices compatible with either wired or wireless connection with such a smart phone or other non-dedicated (a more general-purpose) or dedicated controller (rendered into a special-purpose machine by the invented software executed by the digital processing or computing element within controller 16).

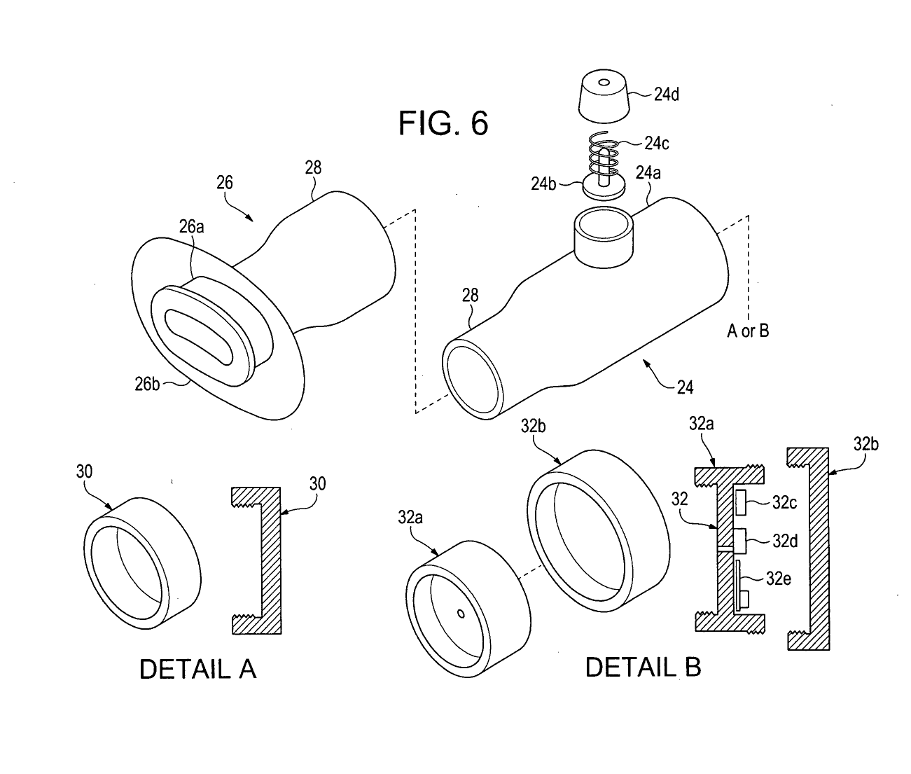

Referring briefly back to FIG. 1, VM device 14 will be understood to include a manometer 22, e.g. a pressure control device and a means of transmitting pressure status during a VM procedure. Any suitable manometer may be used, as may any suitable check valve such as a compact, pressure-adjustable check valve 24 (as shown in FIG. 6 and Details A and B), or of the sort available from Qosina of Edgewood, N.Y., USA. Such may be fixedly adjusted to check the pressure within the VM device until expiratory pressure reaches a defined level at any suitable pressure minimum, e.g. 40 mm Hg. One advantage of using a check valve is that it would build up expiratory pressure until the set-point, and then vent excess pressure, thereby providing the VM device with an easily controlled expiratory pressure and at low cost. If a traditional manometer is used it may be configured to establish, monitor, and indicate when a minimum threshold pressure is reached by the subject's blowing into a preferably disposable mouthpiece 26 (refer briefly to FIG. 6 and to Details A and B) operatively coupled with the manometer and sealingly but removably sealably mounted thereon. Thus, the invention in one embodiment contemplates a reusable pressure monitor and signal transmission means and a disposable manometer 22 including check valve 24 and mouthpiece 26.

Those of skill in the art will appreciate that the manometer 22 may be used by a clinician or attendant or the subject himself or herself to ensure that the VM is properly performed to ensure a reliable diagnostic result. Those of skill will also appreciate that manometer 22 and/or controller 16 may be equipped to operate an analogue or digital readout of the pressure within VM device 14, the gauge indicating the pressure level numerically or by the use of color-coded areas (e.g. red for out-of-bounds and green for in-bounds). Alternatively, manometer 22 and/or controller 16 may be equipped to operate a tone generator that indicates by tonal changes when the subject's VM is within a prescribed and useful range. Those of skill also will appreciate that manometer 22 and/or controller 16 may be equipped to time the operation of a check valve 24 so that it automatically restricts flow for a defined period of time, e.g. fifteen seconds, and then vents to atmosphere to end the VM interval. Further, the manometer may provide pressure input to the controller, e.g. the PC, which will then allow for display and optional recordation of the pressure data, thereby allowing for correlation of a VM's duration and quality. In addition, this time correlation enables step 208, 308, 408, and 908 in FIGS. 2, 3, 4, and 9, respectively. In other words, the coupling of the controller 16 with the VM device 14 enables the controller to determine when the subject's VM started and ended, allowing for all disclosed calculations without intervention from the clinician.

Such subject data in raw, tabulated, and/or graphic form may be archived in any suitable form, and/or may be locally networked and/or shared with health care provider colleagues and staff members. The Internet may be used assuming controller 14, e.g. a PC or other capable device, is so equipped. The data also may be used to generate a printed, aggregated-data subject report, as described below by reference to FIGS. 8A and 8B.

Those of skill in the art will appreciate that at least one useful and novel method of analyzing a subject's cardiac flow or pulsatility response characteristics is enabled by invented system 10. Other analysis methods nevertheless are contemplated as being within the spirit and scope of the invention.

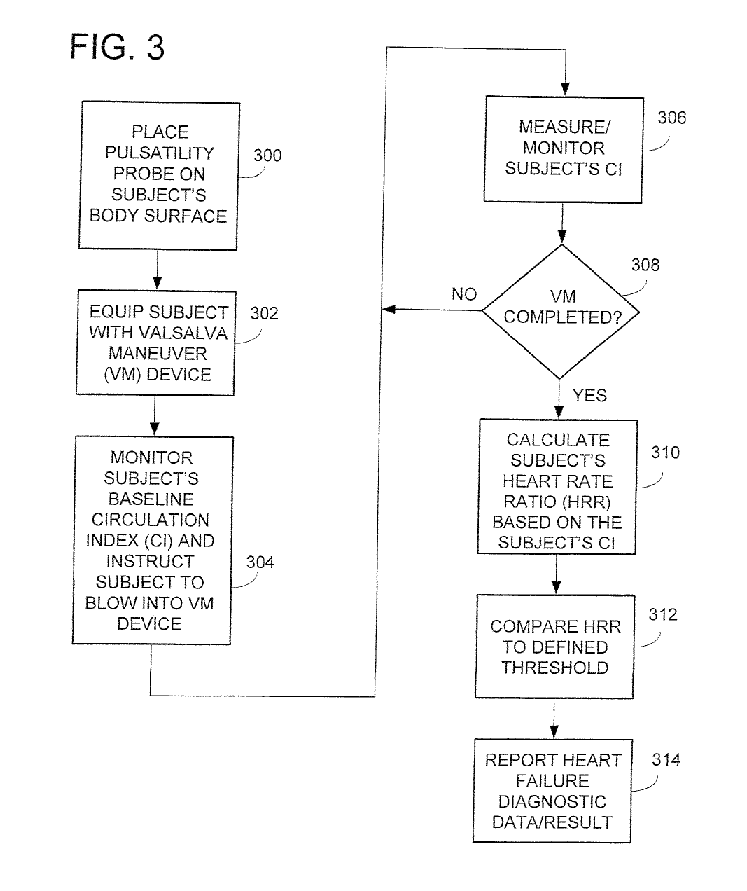

Turning then to FIG. 3, an alternative embodiment of the invented method for analyzing a subject's susceptibility to heart failure is described. Broadly speaking, this method involves estimating the subject's heart rate ratio (HRR). In general, this method includes steps 300-308 and 314 that are identical respectively to steps 200-208 and 214 described above. In particular, the alternative method involves estimating the subject's heart rate ratio (HRR) at 310 and comparing the same to a defined threshold at 314. Equation 2 below shows the formula for calculating the HRR:

HRR=Max(HR_VM)/AVG(HR_Baseline) (2)

wherein AVG(HR_Baseline) is the average heart rate measured during a defined period of time before the Valsalva maneuver begins, wherein Max(HR_VM) is the maximum heart rate during the Valsalva maneuver, and wherein HRR is the arithmetic ratio or quotient therebetween. Those of skill in the art will appreciate that alternative formulations of heart rate ratio are possible and are contemplated as being within the spirit and scope of the invention.

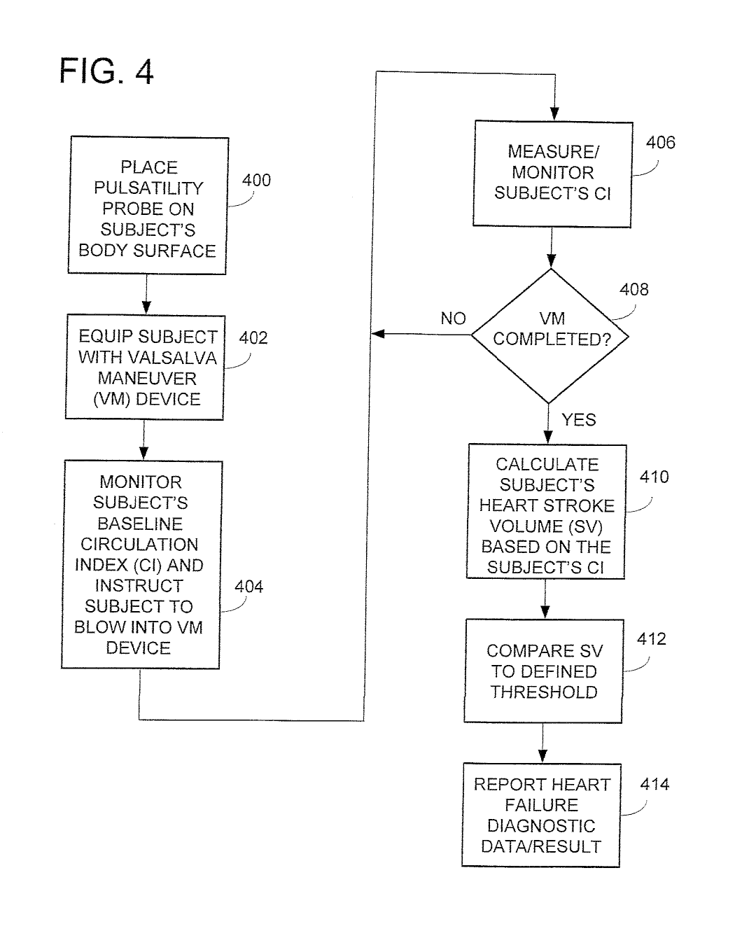

Turning now to FIG. 4, yet another alternative embodiment of the invented method for analyzing a subject's susceptibility to heart failure is described. Broadly speaking, this invented method is aimed at estimating heart stroke volume SV. Generally, this method includes steps 400-408 and 414 that are identical respectively to steps 200-208 and 214 described above. In particular, the alternative method involves estimating the subject's heart stroke volume (SV) at 410 and comparing the same to a defined threshold at 414. Equation 3 below shows the formula for estimating the cardiac stroke volume, in accordance with one embodiment of the invention:

SV=HRR*FiF (3)

wherein HRR and FiF are as previously defined and wherein SV is the arithmetic product thereof. Those of skill in the art will appreciate that alternative methods of estimating SV are contemplated (e.g., the addition of the two components) as being within the spirit and scope of the invention.

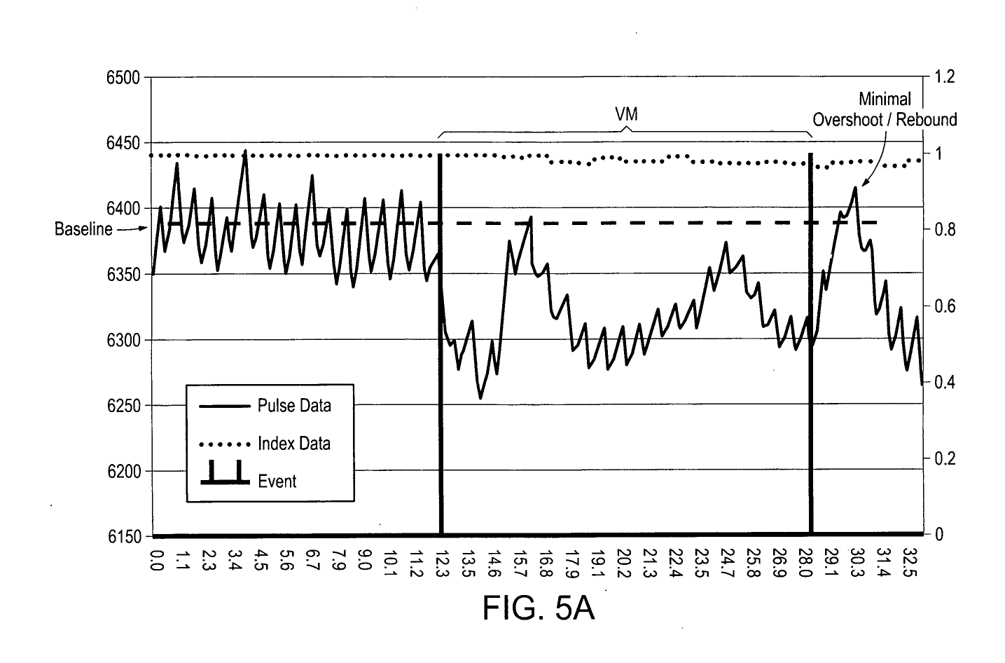

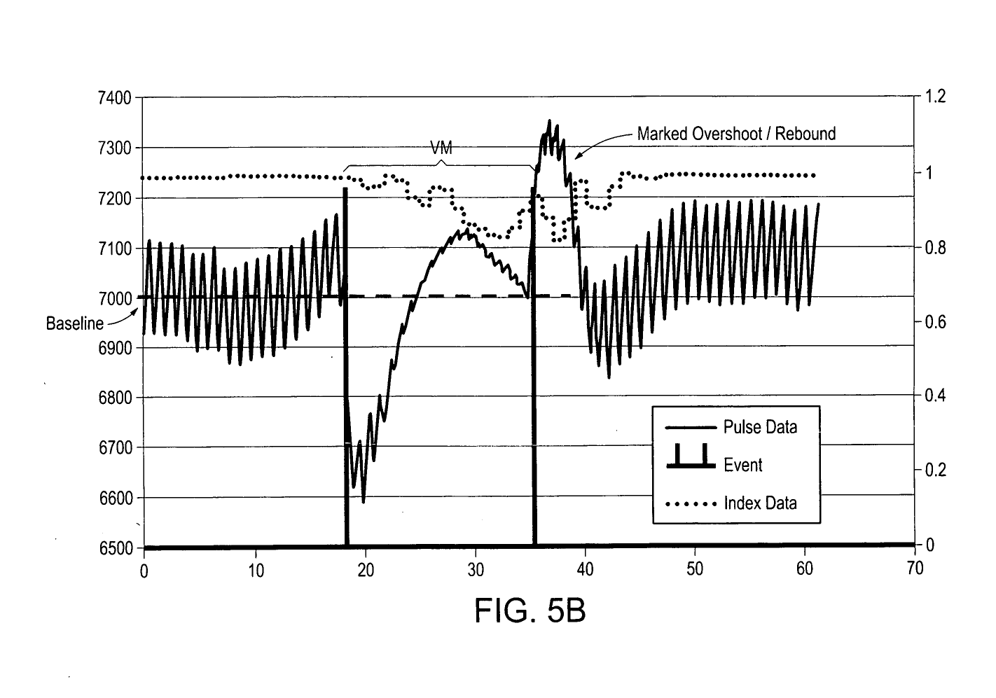

Referring now to FIGS. 5A and 5B, superimposed pulse and circulation index (CI) plots illustrate the contrast between heart failure (FIG. 5A) and healthy (FIG. 5B) subjects. Both graphs have double vertical magnitude axes, the left of which is Relative Blood Volume (arbitrary units) and the right of which is circulation index (CI) (a unitless parameter). Both graphs have horizontal time axes in seconds or tenths of seconds. FIG. 5A shows a substantially flat CI response, while FIG. 5B shows an easily contrasted and substantially more robust and dynamic CI response, during the VM procedure. Additionally, FIG. 5A shows a pulse waveform during the VM that is below the subject's baseline response and fails to overshoot, or Rebound, while FIG. 5B shows a pulse waveform response that is much more dynamic and that exceeds the baseline immediately following the VM interval, or significant Rebound.

The heavy dotted line of FIG. 5A shows the baseline pulse signal prior to the commencement of the VM. As can be seen in the drawing, there is minimal overshoot for a limited time duration after the release of the VM (Rebound). This trait of lacking Rebound is characteristic of heart failure subjects. The heavy dotted line of FIG. 5B shows the baseline pulse signal prior to the commencement of the VM. As can be seen in the drawing, there is significant overshoot for a moderate duration of time after release of the VM. This Rebound is characteristic of healthy subjects.

Moreover, the so-called ‘recovery’ response is significantly different. FIG. 5B shows a marked and distinguishable and thus detectable overshoot in the Rebound response following the release of the VM, while FIG. 5A shows minimal overshoot in the Rebound response. Such heart failure VM response characteristics may also be factored into the diagnostic formulations illustrated herein and described in detail above and below, within the spirit and scope of the invention.

As may be seen in FIG. 5A, the heart-failure subject shows little overshoot at the release of the VM. Literature also supports this lack of Rebound in HF subjects. However, prior art uses invasive means to monitor pressure, as opposed to the invented method of using a non-invasive probe. Conversely, FIG. 5B shows a marked Rebound of a healthy subject. Rebound may be used alone, or in combination with other characteristic metrics such as FiF, HRR, SV, etc., to assess the condition of a patient.

Those of skill in the art will appreciate that the invention lends itself to another method of assessing the condition of a patient. FIG. 5A shows an HR_Baseline (dashed line) of ˜6390 and a minimum (MIN(HR_VM)) during the valsalva maneuver of ˜6260, which is a modest 2.0% change in what will be referred to herein as the steady-state amplitude of the pulsatile waveform. (The steady-state amplitude may be referred to herein as the DC component of the pulsatile response amplitude waveform, or simply, pulse data, which will be understood to ignore frequency components of the waveform above the pulse rate of approximately 1 Hertz (Hz).) FIG. 5A also shows a max/min ratio of ˜6420/˜6260, which could account for a possible rebound, or a ˜2.5% change over the interval of interest.

By way of contrast, in the healthy patient represented by FIG. 5B shows a baseline (dashed line) of ˜7000 and a minimum during the valsalva maneuver of ˜6600, which is a significantly more marked 5.7% change in the steady-state amplitude of the pulsatile waveform. FIG. 5B also shows a max/min ratio of ˜7350/˜6600, or an even more marked ˜10.2% change over the interval of interest. These steady-state amplitude changes shown in FIG. 5B are significantly higher, regardless of how they are calculated, than those steady-state amplitude changes shown for the heart failure (HF) patient represented by FIG. 5A. This observation strongly suggests yet another method of assessing the condition of a patient, since steady-state pulsatile waveform amplitude changes over the interval are readily observed and calculated.

Similar to the calculation of FiF, as depicted in FIG. 2, the controller measures the subject's pulse data during the VM interval. When the VM is completed, the controller calculates the subject's change in DC pulse data based on a ratio of the maximum to minimum DC components during the VM interval (DC Response). Thereafter, the DC Response is compared to a defined threshold level, e.g. between approximately 7% and approximately 27%, preferably approximately 10%. If the subject's DC Response is less than the defined threshold, then the result of the subject's test is interpreted as being positive for heart failure. Finally, the result of the heart failure test is displayed, printed, or otherwise reported, as described and illustrated herein.

Referring next to FIG. 6 and Details A and B thereof, a VM device 14 takes a compact, intra-oral form that integrally embodies some of the elements of the system of FIG. 1. FIG. 6 including Details A and B will be understood to represent an alternative arrangement of parts of a VM device such as VM device 14 made in accordance with a second embodiment the invention. Both VM embodiments include check valve 24 comprising a polymeric body 24a, a seal plunger 24b, a spring 24c, and a threadingly adjustable vent cap 24d, all sealingly assembled as illustrated. The vent cap may alternatively be adhered to the body to prevent movement therebetween. Both VM embodiments also include an elastomeric mouthpiece 26, a tooth positioner 26a, and a lip flange 26b, all preferably integrally molded together, to enable the mouthpiece to be stabilized by the subject during use. Those of skill in the art will appreciate that a distal end of mouthpiece 26 sealingly engages a proximal end of check valve 24 via sealing features 28 that may be molded into or otherwise joined to either or both of the to-be-temporarily-sealingly joined pieces. Those of skill will appreciate that, for example, sealing features 28 may include rigid, ramped, annular, exterior ridges that form an interference fit with a flexible, smooth, annular, interior region of the distal end of mouthpiece 26. Alternatively, the mouthpiece may be affixed to the body via other means including adhesive, simple press-fit, screw/thread, or like means.

Detail A shows a simple end cap assembly 30 configuration for sealingly capping the distal end of body 24a threaded collar that is sealingly walled or capped (see the fragmentary cross-sectional view thereof). Those of skill in the art will appreciate that a low-cost, disposable manometer that does not interface with a controller but that nonetheless provides an adjustable-pressure check valve is provided in accordance with these Detail A teachings of FIG. 6.

Detail B shows a means of capping the distal end of body 24a with an integral manometer pressure sensor 32d capable of wirelessly communicating a pressure reading to a remote device such as controller 16. A capping/manometer means 32 includes a housing 32a with an otherwise sealing barrier wall having an orifice therein and a threaded end cap 32b. In the illustrated cross-sectional view in the lower right corner of FIG. 6 are shown a battery 32c, a pressure transducer 32d sealingly affixed against the orifice within housing 32a, and a wireless printed circuit assembly (PCA) 32e. These electro-tranducer components can be interconnected in any suitable manner, as is known. The housing contains the needed components to power the transducer and to transmit a signal wirelessly to a controller. Accordingly, an integral form of manometer 22 with augmented capability senses the pressure within otherwise sealed housing 28a and wirelessly communicates the same to a controller (e.g. the controller or PC of FIG. 1 that includes a wireless receiver).

Those of skill in the art will appreciate that the intra-oral VM device 14 described above may take alternative forms, yet within the spirit and scope of the invention. For example, a manual manometer gauge may be provided that interfaces via flexible tubing with an intra-oral device having a mouthpiece as otherwise described and illustrated herein. In addition, the manometer 22 may interface directly or not at all with the controller. Any such alternative embodiments of VM device 14 and/or manometer 22 are contemplated as being within the spirit and scope of the invention.

FIG. 7 illustrates a possible screen grab of contents from display 20 in accordance with one embodiment of the invention, the display presentation featuring typical pulsatility waveforms of interest and possible presentation in conjunction with the use of the invented system. The waveform section on the right of the screen shows from top trace down a CI or blood flow waveform graph of a subject with a FiF of 2.3%, a pulse waveform graph absent Rebound, a heart rate waveform graph of the subject with an HR of 78 and a HRR of 1.03, and a pressure graph that highlights the subject's VM with the subject blowing at 40 mm Hg. At the upper left are the subject's vitae, presentation, and test date data. At the lower left is a digital rendition of a manometer indicating that the subject's pressure was properly (in fact it was centered at 40 mm Hg) within the banded area from 30 mm Hg to 50 mmHg for accurate diagnostic test results. Those of skill in the art will appreciate that more, less, different, and differently arranged subject data may be displayed, within the spirit and scope of the invention.

FIGS. 8A and 8B represent alternative printable report formats that are produced by system 10, e.g. via a connected display and/or printer. FIG. 8A is a Valsavagram report on a real but pseudonymous subject whose diagnostic (based on the FiF methodology of FIG. 2 and the HRR methodology of FIG. 3 described above) is positive for heart failure. The waveform that accompanies the subject test results in tabular form shows the characteristic lack of a dramatic and identifiable Rebound overshoot in the pulse or blood flow waveform graph. FIG. 8B is a Valsalvagram report on another real but pseudonymous subject whose diagnostic (based on the FiF methodology of FIG. 2) is negative for heart failure, since this subject's FiF of 28.94% was well above the defined threshold of approximately 13%. Typically, such reports would contain appropriate HIPAA patient security encryption or other governmental or institutional regulatory compliance notices. Those of skill in the art will appreciate that any suitable report format is within the spirit and scope of the invention, including reports that are more or less complete, take a more tabular form or a more graphic form, are differently arranged, etc.

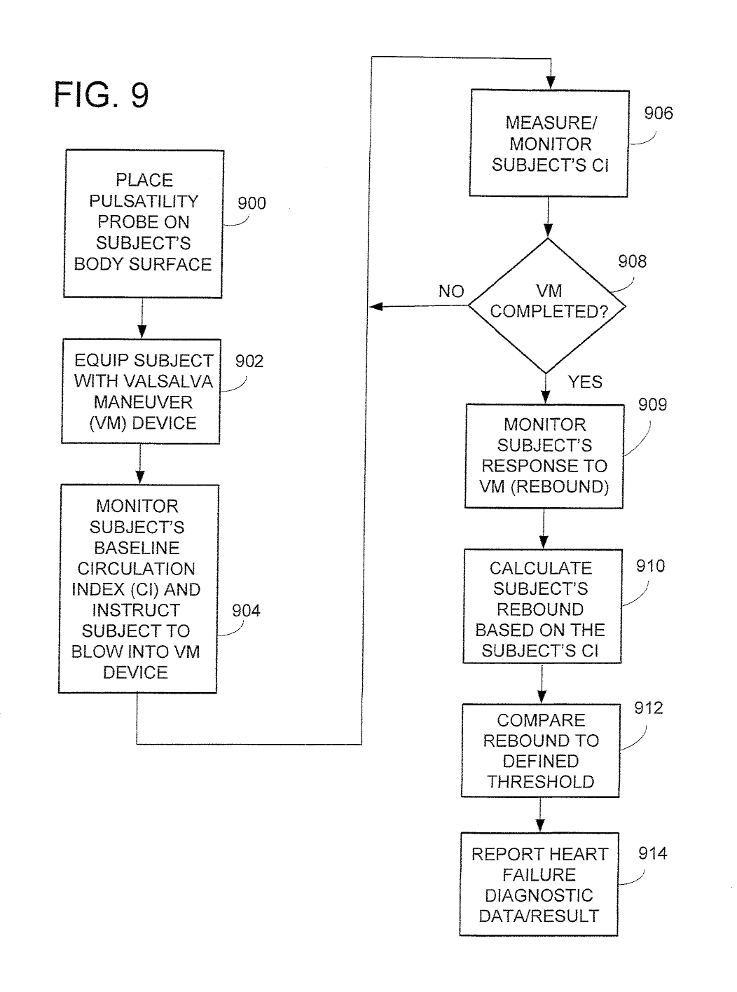

Turning now to FIG. 9, yet another alternative embodiment of the invented method for analyzing a subject's susceptibility to heart failure is described. Broadly speaking, this invented method is aimed at estimating the amount of over-shoot of the subject's pulsatile response immediately following the VM Rebound. Generally, this method includes steps 900-908 and 914 that are identical respectively to steps 200-208 and 214 described above. In addition, the alternative method involves estimating the subject's Rebound at 909 and 910 and comparing the same to a defined threshold at 912. Equation 4 below shows the formula for estimating the Rebound, in accordance with one embodiment of the invention:

$$Rebound = {\sum }_{n = A}^{B} ( {P}_{i} - {P}_{BL} ) \qquad ( 4 )$$

wherein A is the first time the pulse signal exceeds the Baseline Pulse Signal (P_{BL}) and B is the last point above P_{BL} immediately following the VM. Pᵢ is the Pulse signal at each intermediate point, and the difference Pᵢ−P_{BL} is the height above P_{BL}. In effect, Equation 4 is the integration of Pulse Signal points of overshoot immediately following the VM.

Equation 4a below shows an alternative formula for estimating the Rebound, in accordance with another embodiment of the invention:

Rebound=T_{B}−T_{A} (4a)

wherein T_{A} is the first time the pulse signal exceeds the Baseline Pulse Signal (P_{BL}) and T_{B} is the last point above P_{BL} immediately following the VM. In effect, Equation 4a represents the duration of Pulse Signal points of overshoot immediately following the VM. Those of skill in the art will appreciate that alternative methods of estimating Rebound are contemplated as being within the spirit and scope of the invention.

In accordance with the present invention, a healthy subject's Circulation Index (CI) drops markedly—resulting in a substantial FiF—and the healthy subject's HR ratio (HRR) increases, during a VM. Also in accordance with the present invention, a FiF threshold of 0.13 and a HRR threshold of 1.12 have been established below which heart failure is indicated. Thus, it is sensible to multiply the two thresholds together to establish a single metric, an analog for Stroke Volume (SV) with a threshold of 0.15 below which heart failure is indicated. By combining two such metrics, a more accurate assessment of subject status may be derived, as discussed below.

In brief summary and in accordance with the alternative invented methods described and illustrated herein, an accurate, non-invasive heart failure test can be accomplished using relatively low-cost, compact, and portable equipment in less than approximately forty-five seconds per test subject. Invasive, potentially complicated, anesthetized catheterization, post-operative recovery, and follow up are no longer required.

Experiment:

Objective:

The objective was to develop a simple, safe, accurate, portable, non-invasive monitor to detect VM-stressed blood pulsatility or flow anomalies indicative of a subject susceptible to heart failure.

Methodology:

An optical probe that measures infrared light transmission through a finger or toe that was developed in connection with referenced U.S. Pat. No. 7,628,760 B2 was fitted to the toes of a first cohort of thirty-one subjects who presented with unexplained shortness of breath (dyspnea) and a second cohort of twenty-four healthy subjects who were asymptomatic. All subjects were equipped with a VM device and blew into the mouthpiece thereof in accordance with the protocols outlined herein. The patients' pulsatile flow was measured for a continuous period before and after the VM, the pulsatile flow waveforms were time correlated with the VM interval, and the results were interpreted.

Results:

Three of the thirty-one dyspnea cohort were excluded due to protocol variations. Fourteen of the remaining twenty-eight were diagnosed with heart disease via cardiac catheterization. Using a FiF threshold of 13% on both cohorts resulted in a sensitivity, specificity, and accuracy of 71%, 100%, and 92%, respectively. There were four false negatives in the dyspnea cohort, with zero false positives in both cohorts.

Conclusions:

A heart failure diagnostic system in accordance with a first embodiment of the invention described and illustrated herein based achieves a remarkable 92% accuracy using non-invasive means for monitoring a subject's cardiac flow rate change during a Valsalva maneuver. The conventional wisdom that accurate heart failure diagnostics require invasive and expensive catheterization is now in serious question.

The patients from the Experiment disclosed above were analyzed with two approaches: data were collected during the Experiment that allowed for the calculation of both FiF and Rebound as a means for detecting heart failure. Using Rebound alone, the Experiment resulted in a sensitivity, specificity, and accuracy of 93%, 76%, and 80%, respectively. There were nine false positives and one false negative in the dyspnea cohort, with zero false positives and false negatives in the healthy cohort.

As previously mentioned, combining Rebound (or any other metric disclosed herein) with other metrics may result in improved diagnostic results. In the same patient group previously discussed, the Rebound metric was used to set a bi-modal FiF threshold. That is, if Rebound was present, then a 4% FiF threshold was used; if Rebound was absent, then a FiF threshold of 8% was used. The effect of this approach provides a more rigorous FiF threshold (higher) if Rebound is absent, and vice-versa. Using this combined metric approach resulted in a sensitivity, specificity, and accuracy of 71%, 100%, and 92%, respectively. There were four false negatives in the dyspnea cohort, with zero false positives and false negatives in the healthy cohort.

Various embodiments of the invention thus are described in terms of system, method and apparatus for non-invasive but accurate heart failure diagnostic testing of subjects in a portable, compact, and relatively inexpensive form that is readily administered and takes less than a minute.

Those of skill in the art will appreciate that the software architecture and methodologies described and illustrated herein can be implemented in any suitable code by the use of any suitable coding and language tools. For example, any one or more of Python, Java, C#, or C++ are a suitable suite of tools for coding the invented system and controller and device software.

It will be understood that the present invention is not limited to the method or detail of construction, fabrication, material, application or use described and illustrated herein. Indeed, any suitable variation of fabrication, use, or application is contemplated as an alternative embodiment, and thus is within the spirit and scope, of the invention.

It is further intended that any other embodiments of the present invention that result from any changes in application or method of use or operation, configuration, method of manufacture, shape, size, or material, which are not specified within the detailed written description or illustrations contained herein yet would be understood by one skilled in the art, are within the scope of the present invention.

Finally, those of skill in the art will appreciate that the invented method, system and apparatus described and illustrated herein may be implemented in software, firmware or hardware, or any suitable combination thereof. Preferably, the method system and apparatus are implemented in a combination of the three, for purposes of low cost and flexibility. Thus, those of skill in the art will appreciate that embodiments of the methods and system of the invention may be implemented by a general-purpose computer or microprocessor n which purposive instructions are executed, the purposive instructions being stored for execution on a computer-readable medium and being executed by any suitable instruction processor that, in operation, acts as a special-purpose machine performing a special-purpose process.

Accordingly, while the present invention has been shown and described with reference to the foregoing embodiments of the invented apparatus, it will be apparent to those skilled in the art that other changes in form and detail may be made therein without departing from the spirit and scope of the invention as defined in the appended claims.

We claim:

1. A method of predicting heart failure (HF), the method comprising:
measuring the circulation index (CI);
performing a valsalva maneuver (VM);
calculating the change in CI during the VM to produce a fall-in-flow (FiF) value; and
comparing the FiF value to a defined threshold value.

2. The method of claim 1, wherein the measuring, performing, calculating, and comparing steps are implemented in a combination of hardware and software that takes the form of a special-purpose processor executing instructions stored in a memory.

3. The method of claim 2 further comprising:
calculating a change in heart rate by
a) establishing an AVG(HR_Baseline) value equal to the average over a defined time interval before the VM,
b) establishing a HR_VM value equal to the maximum during the VM (Max(HR_VM), and
c) calculating a HR ratio (HRR) of Max(HR_VM) over AVG(HR_Baseline); and
d) comparing the HRR to a threshold value.

4. The method of claim 3 further comprising:
e) estimating heart stroke volume (SV) as a product of HRR and FiF; and
f) comparing the SV to a threshold value.

5. The method of claim 2 further comprising:
calculating a change in heart rate by
a) establishing a HR_Baseline value equal to the average over a defined time interval before the VM,
b) establishing a MIN(HR_VM) value equal to the minimum during the VM, and
c) calculating a HR ratio (HRR) of HR_Baseline over MIN(HR_VM); and
d) comparing the HRR to a threshold value.

6. The method of claim 5 further comprising:
e) estimating heart stroke volume (SV) as a product of HRR and FiF; and
f) comparing the SV to a threshold value.

7. Apparatus for detecting heart failure (HF), the apparatus comprising:
a non-invasive probe configured to illuminate and measure reflective or transmissive light response through a region of a person's body;
a processor coupled with the probe for controlling the illumination and measurement, the processor configured to calculate a circulation index (CI) from the light response data, the processor further configured to calculate a change in CI during the VM to produce a fall-in-flow (FiF) value and to compare the FiF value to a threshold value indicative of heart failure of a person whose extremity is illuminated and monitored.

8. The apparatus of claim 7 further comprising:
a digital manometer coupled with the processor, the manometer configured to indicate barometric pressure caused during an expiration value around the person,
wherein the processor determines based at least in part on the indicated barometric pressure whether a VM by the person meets defined pressure and time requirements.

9. The apparatus of claim 7, wherein the probe is configured to fit on or around an extremity of the person.

10. The apparatus of claim 7, wherein the probe is configured to fit on or in the mouth of the person.

11. The apparatus of claim 10, wherein the probe and manometer are wired or wirelessly coupled with the processor.

12. Apparatus for detecting heart failure (HF), the apparatus comprising:
a non-invasive extremity probe configured to illuminate and measure reflective or transmissive light response through the extremity;
a valsalva maneuver (VM) device configured for a person whose extremity is being monitored to blow thereinto at a defined pressure level for a defined time interval representing a VM;
a processor coupled with the probe for controlling the illumination and measurement, the processor configured to calculate a circulation index (CI) from the light response data, the processor further configured to establish an AVG(HR_Baseline) value equal to the average over a defined time interval before the VM, to establish a HR_VM value equal to the maximum during the VM (Max(HR_VM), to calculate a HR ratio (HRR) of Max(HR_VM over AVG(HR_Baseline), and to compare the HRR to a threshold value indicative of heart failure of the person.

13. The apparatus of claim 12 further comprising:
a digital manometer coupled with the processor, the manometer configured to indicate a barometric pressure caused during an expiration value around the person,
wherein the processor determines whether a VM by the person meets the defined pressure level and the defined time interval based at least in part on the indicated barometric pressure.

14. The apparatus of claim 13, wherein the probe is configured to fit on or around an extremity of the person.

15. The apparatus of claim 14 wherein the probe is configured to fit on or in the mouth of the person.

16. The apparatus of claim 15, wherein the probe and manometer are wired or wirelessly coupled with the processor.

17. A method of predicting heart failure (HF), the method comprising:
applying a non-invasive circulation monitoring apparatus to a patients dermis;
non-invasively measuring a parameter associated with the blood flow;
performing a valsalva maneuver (VM);
calculating a change in the measured parameter during the VM; and
comparing the change in the measured parameter during the VM to a defined threshold value.

18. The method of claim 17, wherein the measured parameter includes a change in the DC component of the pulse data.

19. The method of claim 18, wherein the defined threshold value is in the range of 7% to 27%.

20. The method of claim 17, wherein the measured parameter includes one or more of a fall-in-flow (FiF) value, a HR ratio (HRR), and a heart stroke value (SV) the last being a product of the HRR value and the FiF value.
